(19) United States
(12) Patent Application Publication
(10) Pub. No.: US 2004/0127547 A1
(43) Pub. Date: Jul. 1, 2004

(54) Prodigiosin composition for the treatment of rheumatic arthritis

(76) Inventors: Hwan-Mook Kim, Taejon (KR); Sang-Bac Han, Chungju (KR); Chang-Woo Lee, Taejon (KR); Ki-Hoon Lee, Taejon (KR); Se-Hyung Park, Taejon (KR); Hyoung Chin Kim, Taejon (KR); Young-Kook Kim, Taejon (KR)

Correspondence Address: BIRCH STEWART KOLASCH & BIRCH, PO BOX 747, FALLS CHURCH, VA, 22040-0747, US

(21) Appl. No.: 10/475,130

(22) PCT Filed: Mar. 25, 2002
(86) PCT No.: PCT/KR02/00509

(30) Foreign Application Priority Data
Apr. 19, 2001 (KR) 2001-21191

(51) Int. Cl. A61K 31/4025

(52) U.S. Cl. 514/422

(57) ABSTRACT

The present invention relates to novel use of prodigiosin for the treatment of Rheumatic arthritis and can provide excellent treatment effect to Rheumatic arthritis by treating composition including prodigiosin isolated from Serratia marcescence as an active principle to DBA-1 mouse of collagen-induced Rheumatic arthritis animal model and thereby inhibiting production of internal cytokine which is a important pathogen of Rheumatic arthritis.

### TECHNICAL FIELD

The present invention relates to a composition for the treatment of rheumatic arthritis containing a prodigiosin and more particularly, the present invention relates to a novel use of prodigiosin which is isolated from Serratia marcescens for the treatment of rheumatic arthritis.

### PRIOR ART

Rheumatic arthritis is relatively common disease from which about 1% adult population in all suffers and at least 90% of the patients are females. Its symptoms include stiff joints of both wrists. The diagnosis of this disease can be carried out by a blood test to examine the presence of a rheumatoid factor and an X-ray test to verify the narrowed space between joints or bone abrasion. However, most of the patients shows joint deformation of various types because of the joint destruction over a long period of time. Especially, since the joint deformation in the hands of patients is most severe, there are instances where they are diagnosed as rheumatic arthritis by appearance at a look. Rheumatic arthritis is one of autoimmune diseases and systemic diseases whose etiological cause has not yet been known. This disease symmetrically induces inflammations in various joints, particularly the hands, and consequently, slowly destroys the joints over several to several tens of years. It sometimes invades not only joints but also a variety of organs such as diverse lung, heart, eyes, blood vessels, nerves and the like. Also, this disease affects adults in their before and after thirties, when they lead most active lives, whereby it causes disorders of physical functions and reduction of work ability, leading to deterioration of lives and an enormous economic loss.

Over the past ten years, there has been a rapid progress and critical change in the treatment of rheumatic arthritis. Such a progress is focused on reduction of side effects as low as possible and prevention of joint destruction and development of the disease itself. The conventional anti-inflammatory analgesic agents commonly used in the treatment of rheumatic arthritis inhibit the production metastasis of prostaglandin through an enzyme, so called as COX (cyclooxygenase), in cells, thereby showing antiphlogistic and analgesic effects. There are two types of COX enzymes, COX-1 and COX-2. COX-1 participates extensively in production of prostaglandins in various human organs and tissues of the normal state, that is, the gastrointestinal tract or kidney, as well as inflamed areas. On the other hand, COX-2 acts only in inflamed areas. Existing, commercially available anti-inflammatory analgesic agents inhibit simultaneously COX-1 and COX-2 or mainly COX-1. Therefore, it is known that they inhibit prostaglandins which is indispensable for maintenance of functions of the liver, gastrointestinal tract or kidney, as well as the inflamed tissues, causing numerous side effects. Selective COX-2 inhibitors which have been recently developed have a lowered side effects while maintaining the antiphlogistic and analgesic effects, and thus find their use increased.

The anti-inflammatory and analgesic agents or drugs containing corticosteroid hormone which are widely used as a treating agent for rheumatic arthritis show effective but have defects that they accompany with no small side effects and moreover, cannot fundamentally prevent the progress of rheumatic arthritis. For these reasons, though rheumatic arthritis is temporarily treated through administration of an anti-rheumatic drug, the administration cannot be suspended but a constant dose should be continuously administered. Thus, for patients in such a case, there is a demand for introduction of a novel immunomodulator. Recently, centering around United States, leflunomide, cyclosporine of a low dose and the like have been demonstrated for their effects as a representative drug for the treatment of rheumatic arthritis. As it is known that rheumatic arthritis is developed by extensive and abnormal stimulation of immune cells, attempts are made to develop a novel immunomodulator, perceiving that curative effects can be obtained by intercepting the interaction between immune cells. Rheumatic arthritis is known as a autoimmune inflammatory disease developed by abnormality of immune cells. Immune response of human involves mainly macrophages and lymphocytes. When a foreign substance, particularly arthritogenic factor enters into the human body, antigen presenting cells deals with the factor, that is, by binding epitope fragments (peptides) to HLA gene on the cell surface (peptide-gene complex) and delivering the information to antigen specific T-lymphocytes. Rheumatic arthritis is a disease developed when a particular genetic factor, that is, HLA-DR4 or DR1 in human binds to a foreign substance with strong affinity for the genetic factor, that is, arthritogenic factor and then, binds to a T-cell receptor specific to this antigen, upon which immune response occurs abnormally and excessively to give a signal stimulating each other or lead to secretion of cytokine, thereby calling other immune cells in and amplifying immune response, attacking joints. Therefore, the development of an immunosuppresive capable of inhibiting immune response specific to a substance inducing arthritis may maximize permanently the therapeutic effect on arthritis.

Microorganisms belonging to the genus Streptomyces or Serratia produce red substances of pyrollylpyromethene bone structure, including prodigiosin, methacycloprodigiosin, prodigiosene, desmethoxyprodigiosin, prodigiosin 25-C and the like. These substances are known to have antibiotic and anti-malaria effects. Particularly, prodigiosin 25-C are known to have immunosuppresive action.

The isolation method and immunosuppressive effects of prodigiosin was already reported by the present inventors (Korean Patent No. 252197, registered at Jan. 17, 2000; International Journal of Immunopharmacology 20, 1-13, 1997). The structure of prodigiosin is represented by the following formula 1.

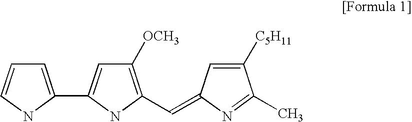

In the above documents, it is described that prodigiosin is an immunosuppressive drug, selective to T-cell, which does not inhibit the antibody production and proliferation of B-cell but to strongly inhibit the proliferation and activity of T-cell and prodigiosin, while not showing toxicity at a concentration where immunosuppression occurs. Prodigiosin has therapeutic effects on autologe transplantation rejection and Type 1 diabetes, as examined by a heterologe transplantation rejection test system and a diabetes test system using NOD mouse. The present inventors have demonstrated the therapeutic effects of prodigiosin isolated from Serratia marcescence on diabetes (Korean Patent Application No. 2000-7139, filed on Feb. 15, 2000). In the above application, it is described that diabetes crisis of the NOD mouse was completely suppressed by intraperitoneally administering prodigiosin at 10 mg/kg every other day. Also, prodigiosin suppressed insulitis by inhibiting lymphocyte infiltration into pancreatic islet. Meanwhile, prodigiosin does not show toxicity even when administered to animal at 10 to 30 mg/kg for 16 weeks, which suggests that prodigiosin is a highly probable candidate for a novel immunosuppressive. Especially, the therapeutic effect of prodigiosin on rheumatic arthritis has not yet been reported.

The present inventors have made the present invention in view of the above problems, and completed the invention by examining therapeutic effects of a composition comprising prodigiosin isolated from Serratia marcescence as an active ingredient on rheumatic arthritis.

Accordingly, it is an object of the present invention to provide prodigiosin composition for treatment of rheumatic arthritis.

### DISCLOSURE OF THE INVENTION

To achieved the object, the present inventors administered a composition comprising prodigiosin isolated from Serratia marcescence as an active ingredient to DBA/1 mice, model animal with collagen-induced rheumatic arthritis, at the early stage of the disease or at the stage in progress and then measured the therapeutic effects and capability to inhibit the production of cytokines, thereby examining the pharmacological mechanism of progidiosin on rheumatic arthritis.

The therapeutic effects of prodigiosin on rheumatic arthritis were verified using an animal model with collagen-induced rheumatic arthritis. In order to measure therapeutic effects of prodigiosin on the outset conditions and early symptoms of rheumatic arthritis, an experiment in which prodigiosin was administered only at the early stage of the disease was carried out. Also, in order to measure therapeutic effects of prodigiosin on progressive aggravated conditions, an experiment in which prodigiosin was administered after the disease had commenced was carried out. In order to examine the pharmacological mechanism of prodigiosin, the expression level of cytokine, a important pathological factor inducing rheumatic arthritis was measured.

Prodigiosin used in the present invention was isolated from Serratia marcescence B-1231, deposited as KCTC 0386BP, according to the method disclosed in Korean Patent No. 252197 invented by the present inventors. Although in the present invention, prodigiosin was administered intraperitoneally at a concentration of 10 mg/kg every other day, the specific dose, mode and time of administration can be varied according to conditions of rheumatic arthritis.

### BRIEF DESCRIPTION OF THE DRAWINGS

The above objects, features and advantages of the present invention will become more apparent from the following detailed description when taken in conjunction with the accompanying drawings, in which:

FIG. 1 is a graph showing the crisis levels of rheumatic arthritis in a DBA/1 mouse with collagen-induced rheumatic arthritis, which were administered intraperitoneally with 10 mg/kg of prodigiosin every other day from the 29^{th} day to 65^{th} day after starting the experiment and a control DBA/1 mouse, not treated with prodigiosin, according to time;

FIG. 2 is a graph showing the crisis levels of rheumatic arthritis in a DBA/1 mouse with collagen-induced rheumatic arthritis, which were administered intraperitoneally with 10 mg/kg of prodigiosin every other day from the 29^{th} day to 36^{th} day after starting the experiment and a control DBA/1 mouse, not treated with prodigiosin, according to time;

FIG. 3 is a graph showing the crisis levels of rheumatic arthritis in a DBA/1 mouse with collagen-induced rheumatic arthritis, which were administered intraperitoneally with 10 mg/kg of prodigiosin every other day from the 36^{th} day to 64^{th} day after starting the experiment and a control DBA/1 mouse, not treated with prodigiosin, according to time;

FIG. 4 shows microscopic photographs of the ininter phalangeal joint (A) and knee joint (B) of DBA/1 mouse, which were administered intraperitoneally with 10 mg/kg of prodigiosin every other day from the 29^{th} day to 36^{th} day after starting the experiment and the ininter phalangeal joint (C) and knee joint (D) of a control DBA/1 mouse, not treated with prodigiosin; and

FIG. 5 is a view showing the expression levels of cytokines in the DBA/1 mouse, which were administered intraperitoneally with 10 mg/kg of prodigiosin every other day from the 29^{th} day to 65^{th} day after starting the experiment and a control DBA/1 mouse, not treated with prodigiosin, using RT-PCR.

### DETAILED DESCRIPTION OF THE PREFERRED EMBODIMENT

Now, specific embodiments of the present invention will now be described by the following examples. However, it should be understood that the scope of the present invention is not limited thereto.

### EXAMPLES

### Example 1

### Preparation of Animal Model with Collagen-Induced Rheumatic Arthritis

6 week-old male DBA/1 mouse supplied from Charles River Japan (Japan) was adapted until 8 to 12 week-old and then, used in the experiment. On the first day of the experiment, the mouse was administered subcutaneously with 100 μl of collagen/complete freund's adjuvant at its tail. The collagen/complete freund's adjuvant was prepared by mixing a solution of collagen (bovine type II) dissolved in 0.05 M acetic acid at a concentration of 2 mg/ml with complete freund's adjuvant at the same concentration. At 21st day of the experiment, 100 μg of collagen was intraperitoneally administered and at 28^{th} day of the experiment, 40 μg of lipopolysaccharide was intraperitoneally administered to promote arthritis crisis. The crisis level of rheumatic arthritis was examined with the naked eye and expressed as a clinical index. That is, 0=none, 0.5=flushed and swollen toe, 1=mild crisis, 1.5=moderate crisis, 2 or more=severe crisis and joint stiffness. Four legs of the DBA/1 mouse were examined for their levels of arthritis crisis. They showed a crisis level of 0 to 8. The development of rheumatic arthritis was promoted by LPS intraperitoneally administered at 28^{th} day of the experiment and the arthritis lesion was observed with the naked eye at 29^{th} day.

### Example 2

### Therapeutic Effect of Prodigiosin on Rheumatic Arthritis

In order to confirm the therapeutic effect of prodigiosin on rheumatic arthritis, three types of experiments were performed according to the following strategies: (1) 10 mg/kg of prodigiosin was intraperitoneally administered every other day from the 29^{th} day of the experiment, when rheumatic arthritis lesion was developed, to 65^{th} day, the last day of the experiment; (2) 10 mg/kg of prodigiosin was intraperitoneally administered every other day from the 29^{th} day of the experiment, when rheumatic arthritis lesion was developed, to 36^{th} day of the experiment; and (3) 10 mg/kg of prodigiosin was intraperitoneally administered every other day from the 36^{th} day of the experiment, when rheumatic arthritis was in progress, to 65^{th} day, the last day of the experiment.

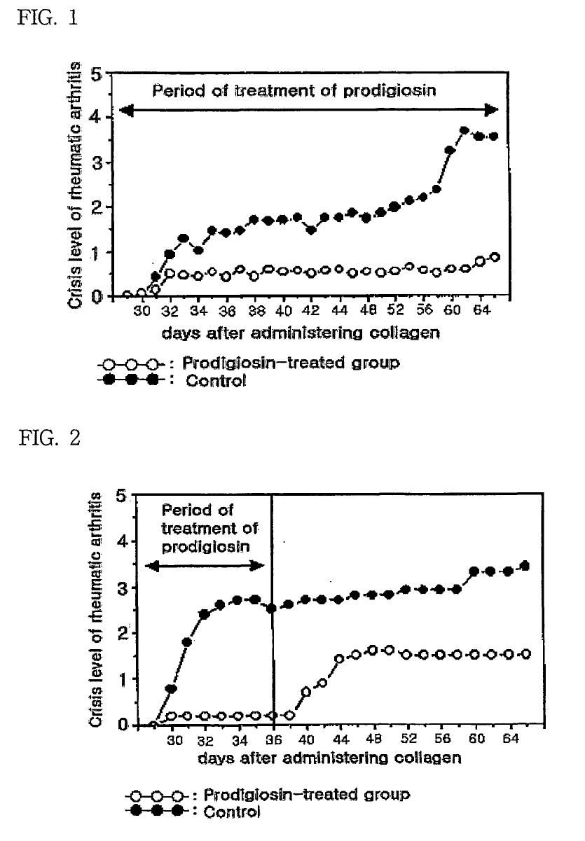

In this example, the DBA/1 mouse with collagen-induced rheumatic arthritis, prepared in Example 1, were administered intraperitoneally with 10 mg/kg of prodigiosin every other day from the 29^{th} day of the experiment to 65^{th} day of the experiment. The DBA/11 mouse treated with prodigiosin according to the above method and the DBA/1 mouse non-treated with prodigiosin were examined for their crisis level of rheumatic arthritis according to time. The results are shown in FIG. 1. The control DBA/1 mouse, not treated with prodigiosin, showed at least 3 of crisis level of rheumatic arthritis (closed circle), while the prodigiosin-treated DBA/1 mouse showed 0.5 or less of crisis level (open circle). From these results, it is noted that prodigiosin is a potent therapeutic agent to strongly inhibit the rheumatic arthritis crisis.

### Example 3

### Therapeutic Effect of Prodigiosin on Early Rheumatic Arthritis

In order to investigate the change after suspension of administration of prodigiosin and the influence of prodigiosin on early rheumatic arthritis, the DBA/1 mouse with collagen-induced rheumatic arthritis, prepared in Example 1, were administered intraperitoneally with 10 mg/kg of prodigiosin every other day from the 29^{th} day of the experiment to 36^{th} day of the experiment. The DBA/1 mouse treated with prodigiosin according to the above method and the DBA/1 mouse non-treated with prodigiosin were examined for their crisis level of rheumatic arthritis according to time. The results are shown in FIG. 2. The control DBA/1 mouse, not treated with prodigiosin, showed at least 3 of crisis level of rheumatic arthritis (closed circle), while the prodigiosin-treated DBA/1 mouse showed 0.5 or less of crisis level by the 38^{th} day of the experiment (open circle). Accordingly, it is noted that prodigiosin has effects preventing and treating rheumatic arthritis. Meanwhile, after suspension of administration of prodigiosin, the crisis level rapidly increased to about 1 but, thereafter, did not show any further serious increase. From these results, it is noted that prodigiosin can not only inhibit the development and early symptoms of arthritis but also prevent the symptoms from growing worse even after suspension of administration. This indicates that the autoimmune response of joints is suppressed by prodigiosin.

### Example 4

### Therapeutic Effect of Prodigiosin on Progressive Rheumatic Arthritis

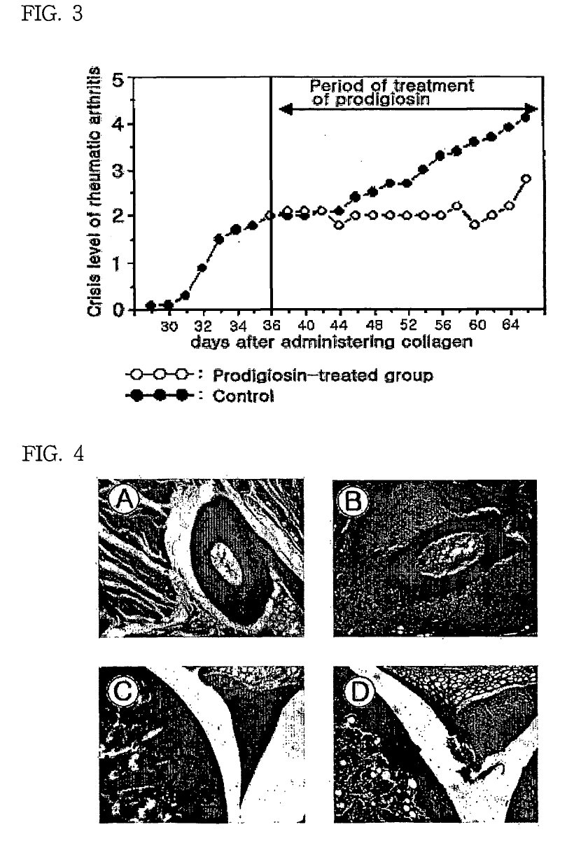

In order to investigate the influence of prodigiosin on rheumatic arthritis which had developed and progressed, the DBA/1 mouse with collagen-induced rheumatic arthritis, prepared in Example 1, were administered intraperitoneally with 10 mg/kg of prodigiosin every other day from the 36^{th} day to 65^{th} day after starting the experiment. The DBA/1 mouse treated with prodigiosin according to the above method and the DBA/1 mouse non-treated with prodigiosin were examined for their crisis level of rheumatic arthritis according to time. The results are shown in FIG. 3. The control DBA/1 mouse, not treated with prodigiosin, showed about 2 of crisis level of rheumatic arthritis at 36 day after starting the experiment, which continuously increased to show about 4 at 65 day after starting the experiment (closed circle), while the prodigiosin-treated DBA/1 mouse showed about 2 of crisis level at 36 day after starting the experiment (open circle), showing no more increase. This indicates that the autoimmune response of joints is suppressed by prodigiosin, thereby preventing progress of rheumatic arthritis.

### Example 5

### Laboratory Test

The ininter phalangeal joints and knee joints from DBA/1 mouse of the prodigiosin-treated group and the control group, not treated with prodigiosin, in Example 3 were subjected to a laboratory test. The ininter phalangeal joints and knee joints were removed on the last day of the administration and stained with hematoxylin and eosin. Their microscopic photographs are shown in FIG. 4. Normal tissues were observed in the ininter phalangeal joint (A) and knee joint (C) from the DBA/1 mouse of the prodigiosin-treated group while rheumatic arthritis lesions were observed in the ininter phalangeal joint (B) and knee joint (D) from the DBA/1 mouse of the control group, not treated with prodigiosin. Also, the lymphocyte infiltration into the joint was observed in the ininter phalangeal joint (B) of the control DBA/1 mouse, not treated with prodigiosin, but not observed in the ininter phalangeal joint (A) of the prodigiosin-treated DBA/1 mouse. Further, the joint destruction was observed in the knee joint (D) of the control DBA/1 mouse, not treated with prodigiosin, but not observed in the knee joint (C) of the prodigiosin-treated DBA/1 mouse, showing normal tissues.

### Example 6

### Inhibition of Production of Cytokine by Prodigiosin

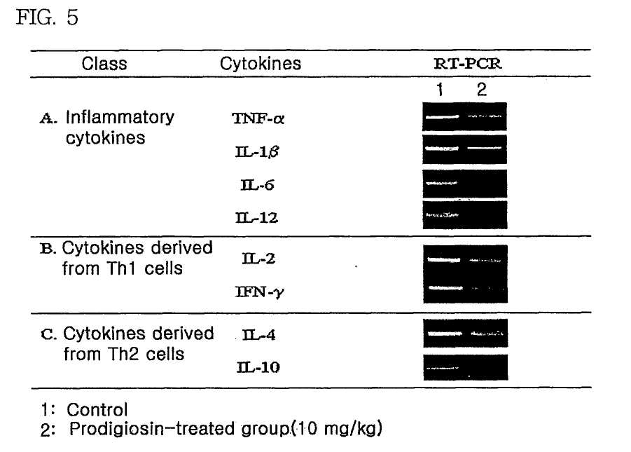

The spleen cells were removed from DBA/1 mouse of the prodigiosin-treated group and the control group, not treated with prodigiosin, in Example 3 and subjected to RNA separation. Then, the expression levels of cytokine were measured using RT-PCR (Reverse transcriptase polymerase chain reaction). The expression levels of inflammatory cytokines such as TNF-α, IL-1β, IL-6, IL-12; cytokines derived from Th1 cells such as IL-2, IFN-γ; cytokines derived from Th2 cells such as IL4, IL-10 were measured. The results are shown in FIG. 5. The administration of prodigiosin reduced the expression levels of all kinds of cytokines above described. From these results, it is noted that therapeutic effect of prodigiosin on rheumatic arthritis can be obtained by inhibiting expression of cytokines which is a critical etiological cause inducing rheumatic arthritis.

### INDUSTRIAL APPLICABILITY

As described in the above Examples, prodigiosin isolated from Serratia marcescence is a rheumatic arthritis drug which can treat early rheumatic arthritis and progressive rheumatic arthritis without side effects. Therefore, the present invention is very useful in the medical industry relating to treatment of patients with rheumatic arthritis.

1. A composition for the treatment of rheumatic arthritis comprising a prodigiosin isolated from Serratia marcescence B-1231 (KCTC 0386BP) as an active ingredient, represented by the following formula 1: [chemical structure]
